(19) United States
(12) Patent Application Publication
(10) Pub. No.: US 2008/0220071 A1
(43) Pub. Date: Sep. 11, 2008

(54) Aqueous Suspensions of Poorly Water-Soluble and Water-Insoluble Active Ingredients and Drying Powder Produced Therefrom

(75) Inventors: Jesper Feldthusen Jensen, Nieder-Olm (DE); Christian Kopsel, Weinheim (DE); Heike Schuchmann, Stutensee (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/994,512

(22) PCT Filed: Jun. 30, 2006
(86) PCT No.: PCT/EP2006/063739

§ 371 Date: Jan. 3, 2008

(30) Foreign Application Priority Data
Jul. 4, 2005 (DE) 10 2005 031 468.6

(51) Int. Cl. A61K 9/14; A23C 21/00; A23K 1/16; A23L 1/30; A61K 31/045; A61K 31/01

(52) U.S. Cl. 424/489; 426/583; 514/729; 514/762

(57) ABSTRACT

Aqueous suspensions comprising: (a) at least one sparingly water-soluble or water-insoluble active agent in the form of nanoparticulate particles; (b) at least one whey component selected from the group consisting of whey proteins, whey protein hydrolysates, and mixtures thereof; and (c) at least one sucrose fatty acid ester having an HLB value of 10 to 18; processes for preparing such aqueous suspensions; powders and oil-miscible compositions prepared therefrom and uses therefore.

The present invention relates to aqueous suspensions comprising
a) at least one sparingly water-soluble or water-insoluble active agent,
b₁) a whey protein and/or
b₂) a whey protein hydrolysate and
c) a sucrose fatty acid ester with an HLB value in the range from 10 to 18.

Numerous active agents suitable for the animal fodder and foodstuff sectors or for pharmaceutical and cosmetic use, for example liposoluble vitamins or carotenoids, but also the natural colorants curcumine or carmine and numerous UV screening agents, can be used, due to their insolubility in water and/or their susceptibility to oxidation, only in the form of specially stabilized compositions. Direct use of the crystalline materials, inter alia for coloring aqueous foodstuffs, as feed additives or as active or effective agents in cosmetic preparations, is generally not possible. The high requirements with regard to bioavailability, coloring properties and dispersibility, in particular in aqueous but also in lipophilic media, can only be met by means of special formulations.

In the direct coloring of foodstuffs, satisfactory colorant yields can only be obtained by compositions in which the active agents, for example carotenoids, are present in finely divided form and protected from oxidation by protective colloids. These formulations used in animal fodder result in an increased bioavailability of the active agents and accordingly, indirectly, in better coloring effects, e.g. in egg yolk or fish pigmentation.

A number of the most varied formulation processes, all having the aim of reducing the crystallite size of the active agents and of bringing to a particles size range of less than 10 μm, are already known from the literature.

Numerous methods, inter alia described in Chimia 21, 329 (1967), WO 91/06292 and WO 94/19411, use in this connection the milling of carotenoids by means of a colloid mill so that particle sizes of 2 to 10 μm are achieved.

In addition, combined emulsifier/spray drying processes exist, such as those disclosed, e.g., in DE-A-12 11 911 or in EP-A-0 410 236.

According to European patent specification EP-B-0 065 193, finely divided pulverulent carotenoid preparations are prepared by dissolving, for example, β-carotene in a volatile water-miscible organic solvent at temperatures of between 50° C. and 200° C., if appropriate under elevated pressure, within a time period of less than 10 seconds. The β-carotene is precipitated from the molecularly disperse solution obtained by immediate rapid mixing with an aqueous solution of a protective colloid at temperatures between 0° C. and 50° C. There is thus obtained a colloidally disperse β-carotene hydrosol with an orange-yellow hue. Subsequently, spray drying the suspension provides a free flowing dry powder which dissolves in water with the formation of a clear suspension colored yellow-orange.

An analogous process for the preparation of finely divided pulverulent carotenoid preparations is disclosed in EP-A-0 937 412 with the use of water-immiscible solvents.

WO 98/26008 relates to the use of a mixture of low molecular weight and high molecular weight protective colloids in the preparation of redispersible xanthophyll-comprising dry powders.

DE 36 11 229 A1 discloses the use of milk or dried milk as protective colloid in the preparation of pulverulent carotenoid compositions.

US 2002/0107292 A1 discloses compositions of lipophilic bioactive active agents together with whey proteins.

It was an object of the present invention to convert hydrophobic sparingly water-soluble or water-insoluble active and effect agents into stable aqueous suspensions or into stable and satisfactorily redispersible dry powders with high bioavailability.

Stable is understood as meaning, for the purposes of the invention, that the formulations are, over a time period and temperature range sufficient for the respective use, stable toward oxidation, light, sedimentation and creaming, inter alia.

The object of the present invention has been achieved by aqueous suspensions comprising
a) at least one sparingly water-soluble or water-insoluble active agent,
b₁) a whey protein and/or
b₂) a whey protein hydrolysate and
c) a sucrose fatty acid ester with an HLB value in the range from 10 to 18.

Mention may preferably be made of aqueous suspensions in which the disperse phase comprises at least one sparingly water-soluble or water-insoluble active agent as nanoparticulate particles. In addition, the dry powders or emulsions, preferably double emulsions, in particular O/W/O emulsions, prepared from the above aqueous suspensions are also in the forefront of the invention.

In this connection, the term “sparingly water-soluble organic active agents” is understood as meaning those compounds which have a solubility in water <5% by weight, preferably <1% by weight, particularly preferably <0.1% by weight, very particularly preferably <0.01% by weight.

Mention may be made, as active agents which are suitable in the context of the present invention for the foodstuff and animal fodder sectors and for pharmaceutical and cosmetic applications, for example, of the following compounds:

Liposoluble vitamins, such as, e.g., the K vitamins, vitamin A and derivatives, such as vitamin A acetate, vitamin A propionate or vitamin A palmitate, vitamin D₂ and vitamin D₃, and vitamin E and derivatives. In this connection, vitamin E represents natural or synthetic α-, β-, γ- or δ-tocopherol, preferably natural or synthetic α-tocopherol, and tocotrienol. Vitamin E derivatives are, e.g., tocopheryl C₁-C₂₀-carboxylates, such as tocopheryl acetate or tocopheryl palmitate.

Polyunsaturated fafty acids, such as, e.g., linoleic acid, linolenic acid, arachidonic acid, eicosapentaenoic acid or docosahexaenoic acid.

Food colorants, such as curcumine, carmine or chlorophyll.

Carotenoids, both carotenes, such as, e.g., β-carotene and lycopene, and xanthophylls, such as, e.g. lutein, astaxanthin, zeaxanthin, capsanthin, capsorubin, cryptoxanthin, citranaxanthin, canthaxanthin, bixin, β-apo-4-carotenal, β-apo-8-carotenal and β-apo-8-carotenic acid ethyl ester.

Phytosterols, coenzyme Q10.

Water-insoluble or sparingly water-soluble organic UV screening substances, such as, e.g., compounds from the group of the triazines, anilides, benzophenones, triazoles, cinnamides and sulfonated benzimidazoles.

Preferred active agents are carotenes, in particular β-carotene or lycopene, and xanthophylls, in particular lutein, astaxanthin and canthaxanthin, and also vitamin A and vitamin E and, from the series of UV screening substances, the family of the triazines, in particular Uvinul T150.

A particularly preferred embodiment of the aqueous suspensions according to the invention is that relating in this connection to aqueous suspensions comprising at least one sparingly water-soluble or water-insoluble active agent chosen from the group of carotenoids, consisting of β-carotene, lycopene, lutein, astaxanthin and canthaxanthin, very particularly preferably lycopene or astaxanthin, as nanoparticulate particles.

When carotenes are used as sparingly water-soluble or water-insoluble active agent, whey proteins in combination with sucrose laurate are preferred while, for aqueous suspensions of xanthophylls, whey protein hydrolysates in combination with sucrose laurate are preferably used.

The solids content in the aqueous suspensions according to the invention ranges from 0.1 to 70% by weight, preferably from 0.5 to 50% by weight, particularly preferably from 10 to 40% by weight.

The mean particle size of the active agent particles in the aqueous suspension ranges, depending on the type of formulation method, from 0.01 to 100 μm, preferably from 0.01 to 10 μm, particularly preferably from 0.01 to 2 μm, very particularly preferably from 0.02 to 1 μm.

The aqueous suspension according to the invention comprises, as component b), a whey protein (b₁) and/or a whey protein hydrolysate (b₂). A spray-dried whey protein isolate with a protein content of at least 80%, particularly preferably with a protein content of at least 95%, is preferably used as whey protein.

Purified enzymatically decomposed whey proteins with a degree of hydrolysis of 3 to 20, particularly preferably with a degree of hydrolysis of 4 to 16 (determined according to the OPA method), are preferably used as whey protein hydrolysate (b₂).

Sucrose fatty acid esters c) with an HLB value in the range from 10 to 18 comprise sucrose stearate, sucrose palmitate, sucrose myristate, sucrose laurate and sucrose oleate. In this connection, the proportion of monoester, for example sucrose monostearate, is each time greater than 55%, preferably in the range from 70 to 85%. Mention may be made, as preferred sucrose fatty acid ester, of sucrose laurate with a proportion of sucrose monolaurate of 75 to 85% and an HLB value of 16.

The amounts of the various components of the suspensions according to the invention are chosen so that the compositions comprise 0.1 to 90% by weight, preferably 1 to 50% by weight, particularly preferably 3 to 30% by weight, very particularly preferably 5 to 25% by weight, of at least one sparingly water-soluble or water-insoluble active agent, 0.1 to 50% by weight, preferably 0.1 to 20% by weight, particularly preferably 1 to 10% by weight, of a whey protein and/or of a whey protein hydrolysate, and 0.1 to 20% by weight, preferably 0.1 to 10% by weight, particularly preferably 1 to 5% by weight, of a sucrose fatty acid ester with an HLB value ranging from 10 to 18. The percentages by weight refer in each case to the dry weight of the suspension.

Preferred aqueous suspensions within the meaning of the present invention comprise a) 0.1 to 90% by weight, preferably 1 to 50% by weight, particularly preferably 3 to 30% by weight, very particularly preferably 5 to 25% by weight, of at least one xanthophyll, b) 0.1 to 50% by weight, preferably 0.1 to 20% by weight, particularly preferably 1 to 10% by weight, of a whey protein and c) 0.1 to 20% by weight, preferably 0.1 to 10% by weight, particularly preferably 1 to 5% by weight, of a sucrose laurate with an HLB value of 16, all percentages being with reference to the dry weight of the aqueous suspensions.

Likewise preferred aqueous suspensions comprise a) 0.1 to 90% by weight, preferably 1 to 50% by weight, particularly preferably 3 to 30% by weight, very particularly preferably 5 to 25% by weight, of at least one carotene, b) 0.1 to 50% by weight, preferably 0.1 to 20% by weight, particularly preferably 1 to 10% by weight, of a whey protein hydrolysate and c) 0.1 to 20% by weight, preferably 0.1 to 10% by weight, particularly preferably 1 to 5% by weight, of a sucrose laurate with an HLB value of 16, all percentages being with reference to the dry weight of the aqueous suspensions.

In addition to the whey proteins and sucrose fatty acid esters, the suspensions according to the invention can additionally comprise one or more protective colloids as component d).

Additional colloids which are suitable according to the invention are advantageously water-soluble or water-swellable protective colloids, such as, for example, bovine, porcine or fish gelatin, in particular acidically or basically decomposed gelatin with Bloom numbers in the range from 0 to 250, very particularly preferably gelatin A 100, A 200, B 100 and B 200, and also low molecular weight enzymatically decomposed gelatin types with the Bloom number 0 and molecular weights of 15 000 to 25 000 D, such as, for example, collagel A and gelitasol P (Stoess, Eberbach, Germany), and also mixtures of these gelatin types, and also starch, dextrin, pectin, gum Arabic, lignosulfonates, chitosan, polystyrenesulfonate, alginates, casein, caseinate, such as sodium caseinate, methylcellulose, carboxymethylcellulose, hydroxypropylcellulose, modified starch, such as starch sodium octenylsuccinate (Capsul, National Starch), plant proteins, such as soy, rice and/or wheat proteins, it being possible for these plant proteins to be present in the derived or native form, or synthetic hydrocolloids, such as polyvinyl alcohols and polyvinylpyrrolidone.

Preferred protective colloids are modified starch, casein and/or sodium caseinate, soy protein and gelatin; casein and/or sodium caseinate are particularly preferred.

The amount of protective colloid additionally used is from 0.1 to 50% by weight, preferably from 1 to 30% by weight, particularly preferably from 2 to 20% by weight, very particularly preferably from 3 to 10% by weight, with reference to the dry weight of the formulation.

The suspensions can additionally also comprise low molecular weight stabilizers, such as antioxidants and/or preservatives, for the protection of the active agents. Suitable antioxidants or preservatives are, for example, α-tocopherol, ascorbic acid, tert-butylhydroxytoluene, tert-butylhydroxyanisole, lecithin, ethoxyquin, methylparaben, propylparaben, sorbic acid or sodium benzoate. The antioxidants or preservatives can be used in amounts of 0.01 to 50% by weight, preferably 0.1 to 30% by weight, particularly preferably 0.5 to 20% by weight, very particularly preferably 1 to 10% by weight, with reference to the dry weight of the formulation.

In addition, the suspensions can also comprise plasticizers for increasing the mechanical stability of a dry powder if appropriate prepared therefrom. Suitable plasticizers are, for example, sugar and sugar alcohols, such as sucrose, maltose, glucose, lactose, trehalose, invert sugar, sorbitol, mannitol, xylitol, glucose syrup, maltodextrin or glycerol. Maltodextrin and/or glucose syrup are preferably used as plasticizers. The plasticizers can be present in amounts of 0.1 to 70% by weight, preferably 10 to 60% by weight, particularly preferably 20 to 50% by weight, with reference to the dry weight of the formulation.

In addition, the suspensions can, apart from the sucrose fatty acid esters, comprise additional low molecular weight surface-active compounds (emulsifiers) in a concentration of 0.01 to 70% by weight, preferably 0.1 to 50% by weight, particularly preferably 0.5 to 20% by weight, with reference to the dry weight of the formulation. Amphiphilic compounds or mixtures of such compounds in particular are suitable as such. In principle, all surfactants with an HLB value of 5 to 20 are suitable. The following are suitable as appropriate surface-active substances: esters of long-chain fatty acids with ascorbic acid, mono- and diglycerides of fatty acids and their oxyethylenated products, esters of monofafty acid glycerides with acetic acid, citric acid, lactic acid or diacetyltartaric acid, polyglycerol fatty acid esters, such as, e.g., the monostearate of triglycerol, sorbitan fatty acid esters, propylene glycol fatty acid esters and lecithin. Ascorbyl palmitate is preferably used.

The present invention also relates to a process for the preparation of an aqueous suspension of at least one sparingly water-soluble or water-insoluble active agent by suspending one or more sparingly water-soluble or water-insoluble active agents a) in an aqueous molecularly disperse or colloidally disperse solution comprising b₁) a whey protein and/or b₂) a whey protein hydrolysate and c) a sucrose fatty acid ester with an HLB value in the range from 10 to 18.

In a preferred embodiment of the process according to the invention, the suspending comprises the following steps
a₁) dissolving at least one sparingly water-soluble or water-insoluble active agent in one or more water-miscible organic solvents or in a mixture of water and one or more water-miscible organic solvents or
a₂) dissolving at least one sparingly water-soluble or water-insoluble active agent a) in one or more water-immiscible organic solvents,
b) mixing the solution obtained according to a₁) or a₂) with an aqueous molecularly disperse or colloidally disperse solution comprising b₁) a whey protein and/or b₂) a whey protein hydrolysate and c) a sucrose fatty acid ester with an HLB value in the range from 10 to 18, the hydrophobic phase of the sparingly water-soluble or water-insoluble active agent being produced as nanodisperse phase, and
c) separating the organic solvent.

The water-miscible solvents used in step a₁) are especially water-miscible thermally stable volatile solvents comprising only carbon, hydrogen and oxygen, such as alcohols, ethers, esters, ketones and acetals. It is advisable to use those solvents which are at least 10% miscible in water, exhibit a boiling point of less than 200° C. and/or have less than 10 carbon atoms. Use is made particularly preferably of methanol, ethanol, n-propanol, isopropanol, 1-methoxy-1,2-butanediol, 1-(n-propoxy)-1,2-propanediol, tetrahydrofuran or acetone.

The term “a water-immiscible organic solvent” means, within the meaning of the present invention, an organic solvent with a solubility in water at standard pressure of less than 10%. In this connection, possible potential solvents are, inter alia, halogenated aliphatic hydrocarbons, such as, e.g., methylene chloride, chloroform and carbon tetrachloride, carboxylates, such as dimethyl carbonate, diethyl carbonate, propylene carbonate, ethyl formate, methyl acetate, ethyl acetate or isopropyl acetate, and ethers, such as methyl tert-butyl ether.

Mention may also be made according to the invention, as a water-immiscible organic solvent, of the oils mentioned at the beginning.

Preferred water-immiscible organic solvents are the following compounds from the group consisting of dimethyl carbonate, propylene carbonate, ethyl formate, ethyl acetate, isopropyl acetate and methyl tert-butyl ether.

Use is made, as particularly preferred solvent for the dispersing/suspending step, of at least one water-miscible organic solvent or a mixture of water and at least one water-miscible organic solvent, very particularly preferably isopropanol or acetone.

In an advantageous embodiment of the abovementioned process according to the invention, in step a), the molecularly disperse solution of at least one sparingly water-soluble or water-insoluble active agent is prepared at temperatures of greater than 30° C., preferably between 50° C. and 240° C., in particular from 100° C. to 200° C., particularly preferably from 140° C. to 180° C., if appropriate under pressure, and, immediately afterward, in step b), treated with the aqueous solution of the protective colloid, a mixing temperature of 35° C. to 120° C. being established.

In the process, the solvent component is transferred into the aqueous phase and the hydrophobic phase of the active agent or agents is produced as nanodisperse phase.

With regard to a more detailed description of the process and equipment for the abovementioned dispersing, reference is made here to EP-B-0 065 193.

The invention also relates to a process for the preparation of a dry powder comprising at least one sparingly water-soluble or water-insoluble active agent as nanoparticulate particles, wherein the aqueous suspensions described above are freed from water and dried.

The conversion to a dry powder can in this connection be carried out, inter alia, by spray drying, spray cooling, freeze drying or drying in a fluidized bed, if appropriate also in the presence of a coating material. Corn starch or silica gel, inter alia, are suitable as coating materials.

In a preferred embodiment of the abovementioned process, the suspension of at least one sparingly water-soluble or water-insoluble active agent prepared is milled before the conversion to a dry powder.

In this connection, the milling can be carried out in a way known per se, e.g. with a ball mill. In this connection, milling is carried out, according to the type of mill used, for a sufficient period of time for the particles to exhibit a mean particle size D[4,3], determined via Fraunhofer diffraction, of 0.1 to 100 μm, preferably 0.2 to 50 μm, particularly preferably 0.2 to 20 μm, very particularly preferably 0.2 to 5 μm, in particular 0.2 to 0.8 μm. The term “D[4,3]” describes the volume-weighted mean diameter (see manual of the Malvern Mastersizer S, Malvern Instruments Ltd., UK).

Further details with regard to the milling and the equipment used for this are found, inter alia, in Ullmann's Encyclopedia of Industrial Chemistry, Sixth Edition, 2000, Electronic Release, Size Reduction, Chapter 3.6.: Wet Grinding, and in EP-A-0 498 824.

In a particularly preferred embodiment of the process according to the invention for the preparation of one of the abovementioned dry powders,
a) at least one sparingly water-soluble or water-insoluble active agent a) is dissolved in a water-miscible organic solvent or a mixture of water and a water-miscible organic solvent at temperatures of greater than 30° C.,
b) the solution obtained is mixed with an aqueous molecularly disperse or colloidally disperse solution comprising b₁) a whey protein and/or b₂) a whey protein hydrolysate and c) a sucrose fatty acid ester with an HLB value in the range from 10 to 18, and
c) the suspension formed is converted to a dry powder.

It is particularly preferred in this connection for the suspension in process step b) to additionally comprise, as component d), casein and/or caseinate.

The invention also relates to pulveruient compositions of at least one sparingly water-soluble or water-insoluble active agent which can be obtained according to any of the abovementioned processes.

The invention likewise relates to a process for the preparation of an oil-miscible composition in the form of a double dispersion comprising at least one sparingly water-soluble or water-insoluble active agent, wherein the aqueous suspensions described at the start are emulsified in oil.

In this connection, a water-in-oil emulsion is formed, if appropriate with use of an emulsifier, in which the water phase comprises nanoparticles, stabilized by protective colloid, of at least one sparingly water-soluble or water-miscible organic UV screening substance. If emulsifiers are required, those W/O emulsifiers known per se with an HLB value of less than 10, in particular from 2 to 6 (cf. H. P. Fiedler, Lexikon der Hilfsstoffe für Pharmazie, Kosmetik und angrenzende Gebiete [Dictionary of Auxiliaries for Pharmaceuticals, Cosmetics and Related Fields], 1996, pages 753 ff) are suitable. Typical representatives of this category of emulsifiers are partial fatty acid esters of polyvalent alcohols, e.g. glycerol monostearate or mixtures of mono-, di- and triglycerides, partial fatty acid esters of sorbitan and/or, preferably, fatty acid esters of polyglycerol, such as, for example, polyglycerol polyricinoleate, which are used in a concentration of 10 to 1000% by weight, preferably 100 to 900% by weight, particularly preferably 400 to 800% by weight, with reference to the active agent or agents.

The dispersant can be both of synthetic, mineral or plant origin and of animal origin. Typical representatives are, inter alia, sesame oil, sunflower oil, corn oil, cottonseed oil, soybean oil or peanut oil, esters of medium-chain vegetable fatty acids, and also paraffin oil, glyceryl stearate, isopropyl myristate, diisopropyl adipate, cetearyl 2-ethyl-hexanoate, hydrated polyisobutene, petroleum jelly, caprylic/capric acid triglycerides, microcrystalline wax, lanolin and stearic acid. The amount of the dispersant is generally 30 to 95% by weight, preferably 50 to 80% by weight, with reference to the total weight of the finished emulsion.

Emulsifying can be carried out continuously or batchwise.

The physical stability of the double dispersion system, such as, for instance, the sedimentation stability, is achieved by very good dispersing of the water phase in the oil phase, e.g. intensive treatment with a rotor/stator disperser at temperatures of 20 to 80° C., preferably 40 to 70° C., or with a high pressure homogenizer, such as an APV Gaulin, or with a very high pressure homogenizer, such as the Microfluidizer in the pressure range from 700 to 1000 bar. The average diameters of the aqueous-disperse phase which can be achieved thereby are smaller than 500 μm, preferably smaller than 100 μm, particularly preferably smaller than 10 μm, in particular smaller than 1 μm.

The invention also relates to liquid oil-miscible compositions of at least one sparingly water-soluble or water-insoluble active agent which can be obtained according to the abovementioned process, comprising, as double dispersion system, an aqueous-disperse phase with a particle size of less than 500 μm, in which particles, stabilized by protective colloid, of one or more sparingly water-soluble or water-insoluble active agents are present in dispersed form, in an oil as dispersant.

The invention also relates to the use of the abovementioned aqueous suspensions as additive in foodstuffs, food supplements, animal fodder, pharmaceutical compositions and cosmetic compositions.

The invention also relates to the use of the abovementioned pulverulent compositions as additive in foodstuffs, food supplements, animal fodder, pharmaceutical compositions and cosmetic compositions.

The invention also relates to the use of the abovementioned liquid oil-miscible compositions as additive in foodstuffs, food supplements, animal fodder, pharmaceutical compositions and cosmetic compositions.

The invention is described in greater detail below by way of the examples.

### EXAMPLE 1 (MICRONIZING)

Preparation of an astaxanthin dry powder using an emulsifier combination of whey protein (BiPro®, Davisco, U.S.A.) and sucrose laurate (L-1695, Mitsubishi-Kagaku Foods Corp.)

24.00 g of crystalline astaxanthin and 9.60 g of α-tocopherol were suspended in 175 g of an azeotropic isopropanol/water mixture at a temperature of 30° C. in a heatable vessel. The active agent suspension was then heated to 90° C. and, at a flow rate of 2.1 kg/h, mixed continuously with additional isopropanol/water azeotrope with a temperature of 223° C. and a flow rate of 2.73 kg/h, astaxanthin having dissolved at a transpiring mixing temperature of 165° C. at a pressure of 55 bar. This active agent solution was immediately mixed subsequently with an aqueous phase consisting of a solution of 10.00 g of BiPro® (5%, with reference to dry weight), 10.00 g of sucrose laurate (5%, with reference to dry weight), 138.24 g of Glucidex® 47 (Roquette Freres) and 3.16 g of preservative (mixture) in 5905 g of distilled water, in which the pH value was adjusted with 1M NaOH to pH 9.5, at a flow rate of 61.05 kg/h.

The active agent particles produced in the mixing exhibited, in the isopropanol/water mixture, a particle size of 94 nm, at an E1/1 value¹⁾ of 126. ¹⁾ The E1/1 value defines, in this connection, the specific extinction of a 1.0% aqueous dispersion of a 10% by weight dry powder in a 1 cm cell at the absorption maximum.

The active agent suspension was subsequently concentrated on a thin film evaporator to a concentration of ca. 23.3% by weight on a dry basis, and spray dried. The dry powder exhibited an astaxanthin content of 11.6% by weight. The dry powder, redispersed in water, had a particle size of 281 nm and exhibited an E1/1 value of 68.

### EXAMPLE 2 (MICRONIZING)

Preparation of an astaxanthin dry powder using an emulsifier combination of whey protein and sucrose laurate

36 g of crystalline astaxanthin and 14.4 g of α-tocopherol were suspended in 262.64 g of an azeotropic isopropanol/water mixture at a temperature of 30° C. in a heatable vessel. The active agent suspension was then heated to 90° C. and, at a flow rate of 2.10 kg/h, mixed continuously with additional isopropanol/water azeotrope with a temperature of 223° C. and a flow rate of 2.73 kg/h, astaxanthin having dissolved at a transpiring mixing temperature of 165° C. at a pressure of 55 bar. This active agent solution was immediately mixed subsequently with an aqueous phase consisting of a solution of 30.0 g of BiPro® (10%, with reference to dry weight), 15.0 g of L-1695 (5%, with reference to dry weight), 192.36 g of Glucidex® 47 and 4.74 g of preservative (mixture) in 8858.42 g of distilled water, in which the pH value was adjusted with 1M NaOH to pH 9.5, at a flow rate of 61.05 kg/h.

The active agent particles produced in the mixing exhibited, in the isopropanol/water mixture, a particle size of 94 nm, at an E1/1 value of 126.

The active agent suspension was subsequently concentrated on a thin film evaporator to a concentration of ca. 22% on a dry basis, and spray dried. The dry powder exhibited an astaxanthin content of 11.0% by weight. The dry powder, redispersed in water, had a particle size of 242 nm and exhibited an E1/1 value of 87.

### EXAMPLE 3 (MICRONIZING)

Preparation of an astaxanthin dry powder using an emulsifier combination of whey protein, sucrose laurate and an additional protective colloid (Na caseinate, Emulac® NA, Meggle)

36.00 g of crystalline astaxanthin and 14.40 g of a-tocopherol were suspended in 262.64 g of an azeotropic isopropanol/water mixture at a temperature of 30° C. in a heatable vessel. The active agent suspension was then heated to 90° C. and, at a flow rate of 2.10 kg/h, mixed continuously with additional isopropanol/water azeotrope with a temperature of 223° C. and a flow rate of 2.73 kg/h, astaxanthin having dissolved at a transpiring mixing temperature of 165° C. at a pressure of 55 bar. This active agent solution was immediately mixed subsequently with an aqueous phase consisting of a solution of 15.00 g of BiPro® (5%, with reference to dry weight), 15.00 g of L-1695 (5%, with reference to dry weight), 15.00 g of Na caseinate (5%, with reference to dry weight), 192.36 g of Glucidex® 47 and 4.74 g of preservative (mixture) in 8858.42 g of distilled water, in which the pH value was adjusted with 1M NaOH to pH 9.5, at a flow rate of 61.05 kg/h.

The active agent particles produced in the mixing exhibited, in the isopropanol/water mixture, a particle size of 99 nm, at an E1/1 value of 133.

The active agent suspension was subsequently concentrated on a thin film evaporator to a concentration of ca. 37% on a dry basis, and spray dried. The dry powder exhibited an astaxanthin content of 11.5% by weight. The dry powder, redispersed in water, had a particle size of 196 nm and exhibited an E1/1 value of 112.

### EXAMPLE 4 (MILLING)

Preparation of an astaxanthin dry powder using a combination of protective colloid (Na caseinate, Emulac® NA, Meggle) and emulsifier system (sucrose laurate and whey protein)

The following mixture was milled in 596.5 g of water at 47-52° C. (pH=9.5, adjusted with 39.2 g of 1M NaOH):

| | | |
|---|---|---|
| Crystalline astaxanthin | 60.0 | g |
| Emulac ® NA | 43.48 | g (with 8% of water) |
| Dry glucose DE 40 | 256.0 | g |
| BiPro ® | 10.0 | g |
| Sucrose laurate | 10.0 | g |
| Tocopherol | 6.0 | g |
| Sipernat ® D17 (Degussa) | 12.0 | g |

The active agent particles produced in the milling exhibited in water, after various milling times, a particle size and an E1/1 value of:

| Milling time | Particle size (nm) | E1/1 value |
|---|---|---|
| First pass (17 min.) | 330 | 44 |
| 1 hour closed-circuit milling | 266 | 73 |
| 2 hours closed-circuit milling | 230 | 90 |
| 3 hours closed-circuit milling | 213 | 100 |
| 4 hours closed-circuit milling | 202 | 107 |
| 5 hours closed-circuit milling | 191 | 111 |

The dry powder (after spray drying) exhibited an astaxanthin content of 14.38% by weight. The dry powder, redispersed in water, had a particle size of 207 nm and exhibited an E1/1 value of 111.

### EXAMPLE 5 (MICRONIZING—COMPARATIVE EXAMPLE)

Preparation of an astaxanthin dry powder using a combination of protective colloid (soy protein) and emulsifier (ascorbyl palmitate)

48.00 g of crystalline astaxanthin, 1.60 g of ascorbyl palm itate (0.4%, with reference to dry weight) and 20.00 g of α-tocopherol were suspended in 350.00 g of an azeotropic isopropanol/water mixture at a temperature of 30° C. in a heatable vessel. The active agent suspension was then heated to 90° C. and, at a flow rate of 2.10 kg/h, mixed continuously with additional isopropanol/water azeotrope with a temperature of 223° C. and a flow rate of 2.70 kg/h, astaxanthin having dissolved at a transpiring mixing temperature of 165° C. at a pressure of 55 bar. This active agent solution was immediately mixed subsequently with an aqueous phase consisting of a solution of 67.68 g of derived soy protein (20.2%, with reference to dry weight), 187.18 g of lactose and 16.20 g of preservative (mixture) in 10 818.69 g of distilled water, in which the pH value was adjusted with 1M NaOH to pH 9.5, at a flow rate of 60.00 kg/h.

The active agent particles produced in the mixing exhibited, in the isopropanol/water mixture, a particle size of 150 nm, at an E1/1 value of 126.

The active agent suspension was subsequently concentrated on a thin film evaporator to a concentration of ca. 25% on a dry basis, and spray dried. The dry powder exhibited an astaxanthin content of 13.4% by weight. The dry powder, redispersed in water, had a particle size of 220 nm and exhibited an E1/1 value of 111.

Experimental results (bioavailability test with trout, duration of test 8 weeks):

| | Relative bioavailability |
|---|---|
| Example 1: | 116% |
| Example 5 (comparative example) | 100% |

Stability in the food pellet after storing for 8 weeks (at ambient temperature):

| | Retention |
|---|---|
| Example 1: | 97% |
| Example 5 (comparative example) | 93% |

1-23. (canceled)

24. An aqueous suspension comprising: (a) at least one sparingly water-soluble or water-insoluble active agent in the form of nanoparticulate particles having a mean particle size of of 0.02 to 1 μm; (b) at least one whey component selected from the group consisting of whey proteins, whey protein hydrolysates, and mixtures thereof, and (c) at least one sucrose fatty acid ester having an HLB value of 10 to 18.

25. The aqueous suspension according to claim 24, wherein the at least one sucrose fatty acid ester comprises sucrose laurate having an HLB value of 16.

26. The aqueous suspension according to claim 24, wherein the at least one whey component comprise a protein hydrolysate having a degree of hydrolysis of 3 to 20.

27. The aqueous suspension according to claim 24, the suspension having a solids content of 0.1 to 70% by weight.

28. The aqueous suspension according to claim 24, wherein the at least one sparingly water-soluble or water-insoluble active agent is present in an amount of 0.1 to 90% by weight, wherein the at least one whey component is present in an amount of 0.1 to 50% by weight, and wherein the at least one sucrose fatty acid ester is present in an amount of 0.1 to 20% by weight, all percentages by weight based on a dry weight of the suspension.

29. The aqueous suspension according to claim 28, wherein the at least one sparingly water-soluble or water-insoluble active agent comprises a xanthophyll, wherein the at least one whey component comprises a whey protein, and wherein the at least one sucrose fatty acid ester comprises sucrose laurate having an HLB value of 16.

30. The aqueous suspension according to claim 28, wherein the at least one sparingly water-soluble or water-insoluble active agent comprises a carotene, wherein the at least one whey component comprises a whey protein hydrolysate, and wherein the at least one sucrose fatty acid ester comprises sucrose laurate having an HLB value of 16.

31. The aqueous suspension according to claim 28, further comprising at least one water-soluble or water-swellable protective colloid.

32. The aqueous suspension. according to claim 31, wherein at least one water-soluble or water-swellable protective colloid comprises a component selected from the group consisting of casein, caseinates, and mixtures thereof, and wherein the at least one water-soluble or water-swellable protective colloid is present in an amount of 0.1 to 50% by weight.

33. A process for producing an aqueous suspension, the process comprising:
providing a mixture of at least one sparingly water-soluble or water-insoluble active agent in one or more solvents selected from the group consisting of water-miscible organic solvents, a mixture of water and one or more water-miscible organic solvents, and one or more water-immiscible organic solvents;
combining the mixture and an aqueous molecularly disperse or colloidally disperse solution comprising: at least one whey component selected from the group consisting of whey proteins, whey protein hydrolysates, and mixtures thereof; and at least one sucrose fatty acid ester having an HLB value of 10 to 18, to provide a nanodisperse hydrophobic phase of the at least one sparingly water-soluble or water-insoluble active agent; and
separating the one or more solvents.

34. The process according to claim 33, wherein the one or more solvents comprises at least one water-miscible organic solvent or a mixture of water and at least one water-miscible organic solvent.

35. The process according to claim 33, wherein the mixture is prepared at a temperature greater than 30° C., and wherein the mixture and the aqueous molecularly disperse or colloidally disperse solution are combined at a temperature of 35 to 120° C.

36. A process for producing a powder, the process comprising:
providing an aqueous suspension according to claim 24; and
dying the suspension, optionally in the presence of a coating material, into a dry powder.

37. The process according to claim 36, farther comprising milling the suspension particles prior to drying.

38. A process for producing a powder, the process comprising:
providing an aqueous suspension prepared by the process according to claim 33; and
drying the suspension, optionally in the presence of a coating material, into a dry powder.

39. A pulverulent composition prepared by the process according to claim 36.

40. A process for preparing an oil-miscible composition, the process comprising: providing an aqueous suspension according to claim 24; and emulsifying the aqueous suspension in an oil in the presence of an emulsifier.

41. A method comprising: providing an aqueous suspension according to claim 24; and combining the aqueous suspension and a component selected from the group consisting of foodstuffs, food supplements, animal feeds, pharmaceutical compositions, and cosmetic compositions.

42. A method comprising: providing a pulverulent composition according to claim 39; and combining the pulverulent composition and a component selected from the group consisting of foodstuffs, food supplements, animal feeds, pharmaceutical compositions, and cosmetic compositions.

43. A method comprising: providing an oil-miscible composition according to claim 40; and combining the oil-miscible composition and a component selected from the group consisting of foodstuffs, food supplements, animal feeds, pharmaceutical compositions, and cosmetic compositions.
